(19) United States
(12) Patent Application Publication
(10) Pub. No.: US 2021/0169338 A1
(43) Pub. Date: Jun. 10, 2021

(54) APPARATUS AND METHOD FOR ESTIMATING AGING LEVEL

(71) Applicant: SAMSUNG ELECTRONICS CO., LTD., Suwon-si (KR)

(72) Inventors: Eui Seok SHIN, Yongin-si (KR); Sung Mo AHN, Yongin-si (KR); Jin Young PARK, Hwaseong-si (KR); Jun Ho LEE, Incheon (KR); Hyeong Seok JANG, Seoul (KR)

(73) Assignee: SAMSUNG ELECTRONICS CO., LTD., Suwon-si (KR)

(21) Appl. No.: 16/883,415

(22) Filed: May 26, 2020

(30) Foreign Application Priority Data
Dec. 4, 2019 (KR) 10-2019-0159808

(51) Int. Cl. A61B 5/00; G01J 3/44; A61B 5/02

(52) U.S. Cl. CPC A61B 5/0082; G01J 3/4406; A61B 5/0071; A61B 2562/0238; A61B 5/02007; A61B 5/7278; A61B 5/7275

(57) ABSTRACT

An apparatus for estimating an aging level may include: a light emitter configured to emit blue light to an object; a light receiver configured to measure fluorescence emanating from the object; and a processor configured to estimate the aging level of the object based on the measured fluorescence and an aging level estimation model.

### CROSS-REFERENCE TO RELATED APPLICATION(S)

This application claims priority from Korean Patent Application No. 10-2019-0159808, filed on Dec. 4, 2019, in the Korean Intellectual Property Office, the disclosure of which is incorporated herein by reference in its entirety.

### BACKGROUND

### 1. Field

Apparatuses and methods consistent with example embodiments relate to estimating an aging level.

### 2. Description of Related Art

With the aging of body tissues or under long-term exposure to high concentrations of glucose, proteins such as collagen in body tissues or blood vessels become glycated by non-enzymatic reactions. These glycated proteins are called Advanced Glycation End products (AGEs). As the amount of AGEs in the body tissues increases, elasticity of the body tissues is reduced as a result of protein denaturation. That is, when the glucose concentration in the blood remains high for a long period of time, glycation of proteins in the blood vessels is accelerated, reducing elasticity of the walls of blood vessels with glycated proteins, and increasing vascular permeability, thereby leading to an increase in oxidative stress and inflammatory factors in the blood vessels.

Such protein denaturation in the blood vessels may be a factor in increasing the risk of cardiovascular disease such as arteriosclerosis and high blood pressure. Furthermore, the increase in glycated proteins in the blood vessels includes increased glycation of collagen proteins in tissue of the dermal layer. The protein denaturation caused by protein glycation may be estimated by measuring skin fluorescence.

### SUMMARY

According to an aspect of an example embodiment, there is provided an apparatus for estimating an aging level, the apparatus including: a light emitter configured to emit blue light to an object; a light receiver configured to measure fluorescence emanating from the object; and a processor configured to estimate the aging level of the object based on the measured fluorescence and an aging level estimation model.

The processor may correct the measured fluorescence based on either one or both of a first intensity of the blue light emitted from the light emitter and a second intensity of the blue light reflected from the object.

The light receiver may include either one or both of a spectrometer and an image sensor.

The light receiver may further include a high-pass filter configured to pass a light beam having a wavelength of 500 nm or greater, among a plurality of light beams emanating from the object, and wherein the at least one of the spectrometer and the image sensor may be configured to measure the fluorescence based on the light beam having the wavelength of 500 nm or greater.

The light emitter may emit the blue light having a single wavelength or a plurality of wavelengths.

The light emitter comprises a light source configured to emit a light, and a low-pass filter (LPF) configured to pass a wavelength of 500 nm or less of the light emitted from the light source, or a band-pass filter configured to pass a wavelength range of 400 nm to 500 nm of the light emitted from the light source. The light emitter may be further configured to emit the light having the wavelength of 500 nm or less, or the light in the wavelength range of 400 nm to 500 nm, as the blue light.

The aging level estimation model may include a regression model which defines at least one of a first correlation between the fluorescence and a biological age and a second correlation between the fluorescence and the aging level of blood vessels.

The processor may be further configured to estimate either one or both of the biological age and the aging level of blood vessels by using the regression model.

The processor may be further configured to predict either one or both of a risk of cardiovascular disease and a risk of aging-related disease of a user based on the estimated aging level.

The apparatus may further include an output interface configured to output at least one of the estimated aging level, the risk of cardiovascular disease, and the risk of aging-related disease.

According to an aspect of another example embodiment, there is provided a method of estimating an aging level, the method including: emitting blue light to an object; measuring fluorescence emanating from the object; estimating the aging level of the object based on the measured fluorescence and an aging level estimation model.

The estimating the aging level may include correcting the measured fluorescence based on either one or both of a first intensity of the blue light emitted to the object and a second intensity of the blue light reflected from the object.

The method may further include passing a light beam having a wavelength of 500 nm or greater, among a plurality of light beams emanating from the object, and wherein the measuring the fluorescence may include measuring the fluorescence based on the light beam having the wavelength of 500 nm or greater.

The emitting of the blue light may include, by using a light source, emitting blue light having a single wavelength or a plurality of wavelengths.

The emitting of the blue light may include: emitting a light by a light source; passing a wavelength of 500 nm or less of the light emitted from the light source, or passing a wavelength range of 400 nm to 500 nm of the light emitted from the light source; and emitting, as the blue light, the light having the wavelength of 500 nm or less, or the light in the wavelength range of 400 nm to 500 nm.

The method of estimating an aging level may further include predicting either one or both of a risk of cardiovascular disease and a risk of aging-related disease of a user based on the estimated aging level.

In addition, the method of estimating an aging level may further include outputting at least one of the estimated aging level, the risk of cardiovascular disease, and the risk of aging-related disease.

According to an aspect of another example embodiment, there is provided an apparatus for estimating an aging level, the apparatus including: a fluorescence measuring sensor including a plurality of light sources configured to emit blue light to an object, and a spectrometer configured to measure fluorescence emanating from the object; and a processor configured to estimate an aging level based on the measured fluorescence.

The processor may correct the measured fluorescence based on either one or both of a first intensity of the blue light emitted from the fluorescence measuring sensor and a second intensity of the blue light reflected from the object, and may estimate the aging level based on the corrected fluorescence and an aging level estimation model.

The apparatus for estimating an aging level may further include an output interface configured to output the estimated aging level.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for estimating an aging level according to an example embodiment;

FIGS. 2A and 2B are diagrams explaining an aging level estimation model;

FIG. 3 is a block diagram illustrating an apparatus for estimating an aging level according to another example embodiment;

FIG. 4 is a block diagram illustrating an apparatus for estimating an aging level according to yet another example embodiment;

FIG. 5 is a diagram illustrating a structure of a fluorescence measuring sensor of an apparatus for estimating an aging level according to an example embodiment;

FIG. 6 is a flowchart illustrating a method of estimating an aging level according to an example embodiment;

FIG. 7 is a flowchart illustrating a method of estimating an aging level according to another example embodiment;

FIG. 8 is a diagram illustrating an example of a wearable device; and

FIG. 9 is a diagram illustrating an example of a smart device.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘part’ or ‘module’, etc., should be understood as a unit that performs at least one function or operation and that may be embodied as hardware, software, or a combination thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Hereinafter, example embodiments of an apparatus and method for estimating an aging level will be described in detail with reference to the accompanying drawings.

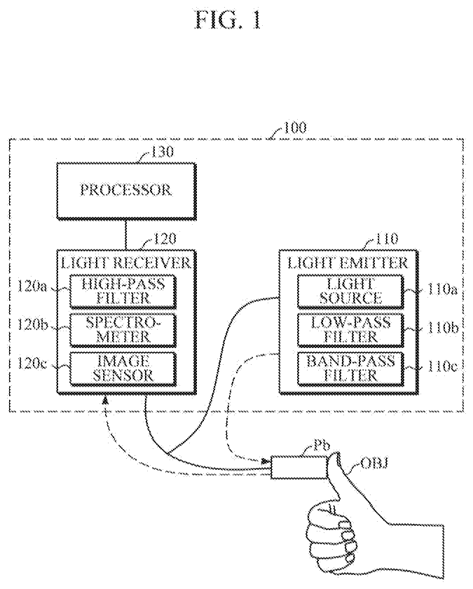

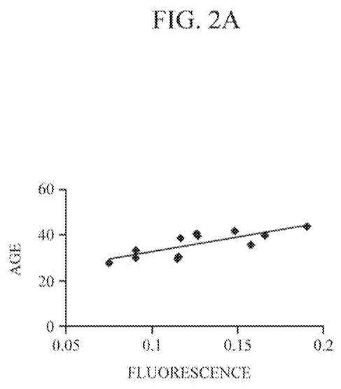

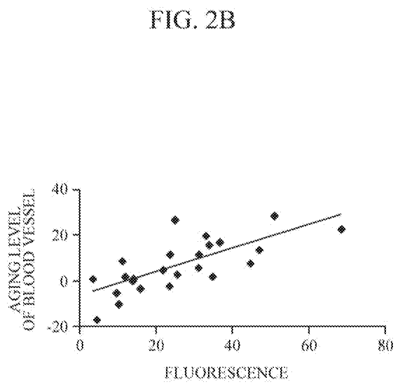

FIG. 1 is a block diagram illustrating an apparatus for estimating an aging level according to an example embodiment. FIGS. 2A and 2B are diagrams explaining an aging level estimation model.

Referring to FIG. 1, the apparatus for estimating an aging level includes a light emitter 110, a light receiver 120, and a processor 130.

The light emitter 110 may include one or more light sources 110a for emitting light when an object OBJ comes into contact with a probe Pb. In this case, the light source 110a may include a light emitting diode (LED), a laser diode (LD), a phosphor, and the like. The light source 110a may emit blue light having a single wavelength (e.g., 470 nm) or a plurality of wavelengths (e.g., wavelength range of 400 nm to 500 nm).

In addition, the light emitter 110 may further include a filter for passing light in a specific wavelength range, which is emitted by the light source 110a. For example, the light emitter 110 may include a low-pass filter (LPF) 110b, and may emit light having a wavelength of 500 nm or less to the object by passing light in a wide wavelength range, which is emitted by the light source 110a, through the low-pass filter 110b. In another example, the light emitter 110 may include a band-pass filter 110c, and may emit light in a predetermined wavelength range of, for example, 400 nm to 500 nm, specifically light having a wavelength of 470 nm, to the object by passing light, emitted by the light source 110a, through the band-pass filter 110c.

Once light, emitted by the light emitter 110, is incident on the object OBJ through the probe Pb, and then is reflected or scattered from the object OBJ to be received through the probe Pb, the light receiver 120 may measure skin fluorescence. In this case, the light receiver 120 may include a spectrometer 120b or an image sensor 120c.

Further, the light receiver 120 may further include a filter for passing light in a specific wavelength range before light, received through the probe PB, enters the spectrometer 120b or the image sensor 120c. For example, the light receiver 120 may include a high-pass filter 120a which passes light in a wavelength range of, from example, 500 nm or more, among light beams emanating from the object.

The processor 130 may estimate an aging level based on the object's fluorescence measured by the light receiver 120. For example, by using a pre-defined aging level estimation model, the processor 130 may estimate the aging level from the measured fluorescence. In this case, the aging level may include a biological age and/or an aging level of blood vessels, and the like, but is not limited thereto.

As illustrated in FIGS. 2A and 2B, the aging level estimation model may be a regression model which defines a correlation between skin fluorescence and a biological age, or a regression model which defines a correlation between skin fluorescence and an aging level of blood vessels. As illustrated herein, the aging level estimation model may be in the form of a linear function, but is not limited thereto, and may be pre-defined using various methods such as non-linear regression analysis, neural network, deep learning, and the like.

A plurality of aging level estimation models may be generated in groups based on a variety of information such as a user's age, sex, race, occupation, stature, body mass index (BMI), smoking status, blood hemoglobin A1c (HbA1c) concentration, health information, and the like. In this case, the processor 130 may select an aging level estimation model suitable for a user from among the plurality of aging level estimation models, and may estimate an aging level by using the selected aging level estimation model.

Further, the processor 130 may correct the fluorescence measured by the light receiver 120, and may estimate an aging level by using the corrected fluorescence and the aging level estimation model. For example, the processor 130 may correct the measured fluorescence based on one or more of an intensity of light incident on the object and an intensity of light reflected from the object. For example, the following Equation 1 is an example of a pre-defined correction equation for correcting fluorescence based on the incident light and the reflected light. However, the equation is not limited to the function.

$${f}_{xm} = \frac{{F}_{xm}}{{Rx}^{{k}_{x}} {Rm}^{{k}_{m}}} \qquad [ Equation 1 ]$$

Herein, fₓₘ denotes the corrected fluorescence, and Fₓₘ denotes the fluorescence measured by the light receiver 120. Further, Rx denotes the intensity of the incident light, Rm denotes the intensity of the reflected light, and kₓ and kₘ are pre-defined correction factors.

In addition, the processor 130 may predict the risk of cardiovascular disease and the risk of aging-related disease by using an aging level estimation result. In this case, the cardiovascular disease refers to conditions affecting the heart and blood vessels, and examples thereof may include hypertension, ischemic heart disease, coronary-artery disease, angina, myocardial infarction, arteriosclerosis, arrhythmia, cerebrovascular disease, apoplexy, and the like.

For example, if the estimated aging level exceeds a threshold value, the processor 130 may predict the risk of cardiovascular disease. In this case, depending on a degree of exceeding the threshold value, the processor 130 may classify the risk into a plurality of categories. The processor 130 may predict the risk of cardiovascular disease and the risk of aging-related disease from the estimated aging level by considering user characteristics. The user characteristic information may include at least one of a user's age, sex, race, occupation, stature, BMI index, smoking status, blood HbA1c concentration, and health information, but is not limited thereto.

For example, with the aging of body tissue, proteins in the body tissue or blood vessels become glycated by non-enzymatic reactions, and autofluorescence of body tissue increases accordingly, such that by comparing the estimated aging level with a reference aging level for an actual age, the processor 130 may predict the risk of cardiovascular disease.

By estimating the aging level and/or predicting the risk of cardiovascular disease/aging-related disease, the processor 130 may perform necessary actions, such as providing guide information so that a user may take actions for reducing the risk of cardiovascular disease.

In addition, the processor 130 may calibrate an aging level estimation model at predetermined intervals or by using an aging level estimation history, a change in user characteristics, and the like.

For example, the processor 130 may obtain a difference between the estimated aging level, which is estimated by using an aging level estimation model, and a reference aging level measured by an external apparatus for measuring skin fluorescence, and if the difference exceeds a pre-defined threshold value, the processor 130 may calibrate the aging level estimation model. Alternatively, based on the aging level estimation history, if a number of times that the difference between the estimated aging level and the reference aging level exceeds the threshold value is equal to or greater than a predetermined number, the processor 130 may determine that calibration is required. In addition, if there is a change in user characteristics, e.g., age, health condition, and the like, the processor 130 may determine that calibration is required. However, the calibration is not limited thereto.

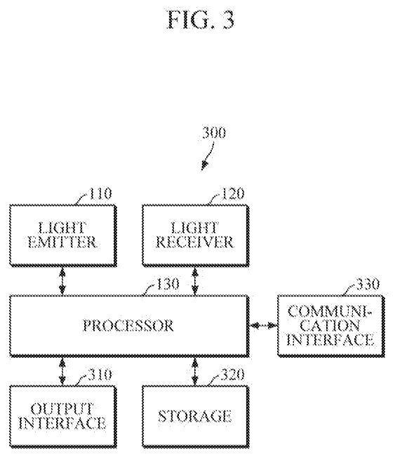

FIG. 3 is a block diagram illustrating an apparatus for estimating an aging level according to another example embodiment.

Referring to FIG. 3, the apparatus 300 for estimating an aging level includes the light emitter 110, the light receiver 120, the processor 130, an output interface 310, a storage 320, and a communication interface 330. The light emitter 110, the light receiver 120, and the processor 130 are described above with reference to FIG. 1, such that detailed description thereof will be omitted.

The output interface 310 may provide information related to an aging level for a user by using an output module under the control of the processor 130. For example, the output interface 310 may output a measured fluorescence, a corrected fluorescence, an estimated aging level, the risk of cardiovascular disease/aging-related disease, predetermined actions in response to the risk of cardiovascular disease/aging-related disease, and the like by various visual/non-visual methods using a display, a speaker, a haptic device, and the like.

The storage 320 may store programs or instructions for operation of the apparatus 300 for estimating an aging level. Further, the storage 320 may store various data processed by the processor 130. For example, the storage 320 may store a variety of information required for estimating an aging level, e.g., an aging level estimation model, a fluorescence correction equation, a reference aging level, a user's characteristic information, and the like. In addition, the storage 320 may store information such as a measured fluorescence value, a corrected fluorescence value, an estimated aging level, and the like.

The storage 320 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like. Further, the apparatus 300 for estimating an aging level may operate an external storage medium, such as web storage and the like, which performs a storage function of the storage 320 on the Internet.

The communication interface 330 may communicate with an external device. For example, the communication interface 330 may transmit, to the external device, information such as user information input by a user, the measured fluorescence value, the corrected fluorescence value, the estimated aging level, the risk of cardiovascular disease/aging-related disease, and the like. Further, the communication interface 330 may receive, from other external device, information such as user information, an aging level estimation model, and the like. In this case, the external device may be medical equipment of a specialized medical institution, an apparatus for measuring fluorescence, a digital TV, a desktop computer, a cellular phone, a smartphone, a tablet PC, a laptop computer, a personal digital assistant (PDA), a portable multimedia player (PMP), a navigation device, an MP3 player, a digital camera, a wearable device, and the like, but the external device is not limited thereto.

The communication interface 330 may communicate with the external device by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WiFi communication, Radio Frequency Identification (RFID) communication, 3G, 4G, and 5G telecommunications, and the like. However, this is merely exemplary and not intended to be limiting.

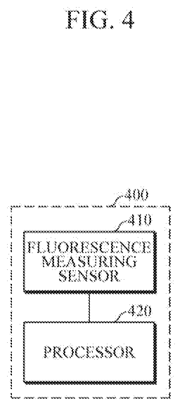

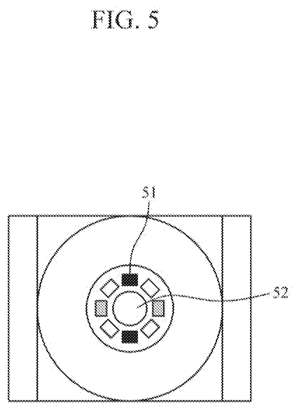

FIG. 4 is a block diagram illustrating an apparatus for estimating an aging level according to yet another example embodiment. FIG. 5 is a diagram illustrating a structure of a fluorescence measuring sensor of an apparatus for estimating an aging level according to an example embodiment.

Referring to FIG. 4, the apparatus 400 for estimating an aging level includes a fluorescence measuring sensor 410 and a processor 420.

The fluorescence measuring sensor 410 may be manufactured in a small size so as to be mounted in a wearable device, a smart device, and the like. For example, the fluorescence measuring sensor 410 may include a plurality of LED light sources and a spectrometer chip which are integrally formed with each other. For example, as illustrated in FIG. 5, a small spectrometer chip 52 may be disposed at the center, and a plurality of LED light sources 51 may be arranged around the small spectrometer chip 52. In this case, the plurality of LED light sources 51, which are arranged around the small spectrometer chip 52, may be arranged in various shapes, such as circle, square, triangle, and the like. All the plurality of LED light sources 51 may emit light having a single wavelength (e.g., 470 nm). Alternatively, each of the plurality of LED light sources 51 may emit light of different wavelengths ranging from 400 nm to 500 nm.

When a user touches the fluorescence measuring sensor 410 with an object for measuring an aging level, the fluorescence measuring sensor 410 may emit light onto the user's object by using the light source 51, and may measure skin florescence, reflected or scattered from the object, by using the spectrometer chip 52. The fluorescence measuring sensor 410 may be electrically connected to the processor 420, and may transmit the measured skin fluorescence to the processor 420.

As described above, the processor 420 may estimate the aging level and/or the risk of cardiovascular disease/aging-related disease based on the fluorescence received from the fluorescence measuring sensor 410. The processor 420 may correct the fluorescence measured by the fluorescence measuring sensor 410, and may estimate the aging level by using the corrected fluorescence and the aging level estimation model.

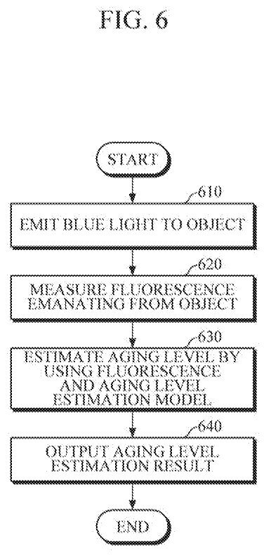

FIG. 6 is a flowchart illustrating a method of estimating an aging level according to an example embodiment. The method of estimating an aging level according to the example embodiment may be performed the aforementioned apparatuses 100, 300, and 400 for estimating an aging level.

The apparatus for estimating an aging level may emit blue light to an object in operation 610. For example, by using a light source 110a which emits light in a wavelength range of 400 nm to 500 nm, the apparatus for estimating an aging level may emit light to the object. Alternatively, by providing a low-pass filter 110b or a band-pass filter 110c on a front surface of the light source 110a, and passing light, emitted by the light source 110a, through the low-pass filter 110b or the band-pass filter 110c, the apparatus for estimating an aging level may allow light in the range of 400 nm to 500 nm, e.g., light having a wavelength of 470 nm, to be incident on the object.

Then, the apparatus for estimating an aging level may measure fluorescence emanating from the object after being reflected or scattered therefrom in operation 620. For example, by using a spectrometer 120b or an image sensor 120c, the apparatus for estimating an aging level may measure fluorescence reflected or scattered from the object. In this case, the apparatus for estimating an aging level may include a high-pass filter 120a on a front surface of the spectrometer 120b or the image sensor 120c, and may allow light, scattered or reflected from the object, to pass through the high-pass filter 120a, so that light having a wavelength of 500 nm or more may be detected by the spectrometer 120b or the image sensor 120c.

Subsequently, the apparatus for estimating an aging level may estimate an aging level in operation 630 by using the fluorescence measured in operation 620 and an aging level estimation model. In this case, the aging level may include a biological age and/or an aging level of blood vessels. The aging level estimation model may be a regression model which defines a correlation between the measured fluorescence and the biological age or aging level of blood vessels.

Next, the apparatus for estimating an aging level may output an aging level estimation result, and may provide the estimation result for a user in operation 640. The apparatus for estimating an aging level may provide the aging level estimation result for the user using various output modules such as a display, a speaker, a haptic device, and the like.

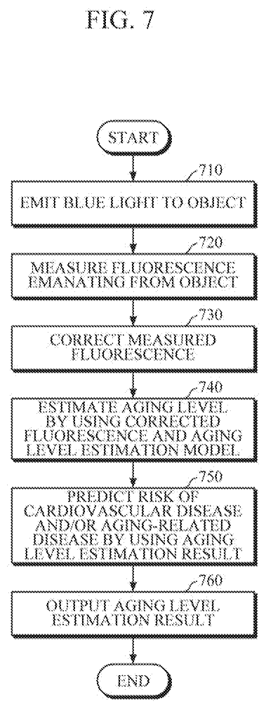

FIG. 7 is a flowchart illustrating a method of estimating an aging level according to another example embodiment. The method of estimating an aging level according to the example embodiment may be performed the aforementioned apparatuses 100, 300, and 400 for estimating an aging level.

The apparatus for estimating an aging level may emit blue light to an object in operation 710. For example, by using a light source 110a which emits light in a wavelength range of 400 nm to 500 nm, or by providing a low-pass filter 110b or a band-pass filter 110c on a front surface of the light source 110a, the apparatus for estimating an aging level may allow light in the range of 400 nm to 500 nm to be incident on the object.

Then, the apparatus for estimating an aging level may measure fluorescence emanating from the object after being reflected or scattered therefrom in operation 720. In this case, the apparatus for estimating an aging level may include a high-pass filter 120a on a front surface of the spectrometer 120b or the image sensor 120c, to pass light having a wavelength of 500 nm or more.

Subsequently, the apparatus for estimating an aging level may correct, in operation 730, the fluorescence measured in operation 720. For example, the apparatus for estimating an aging level may correct the measured fluorescence based on an intensity of light incident on the object and an intensity of light reflected from the object.

Next, the apparatus for estimating an aging level may estimate an aging level, including a biological age or an aging level of blood vessels, and the like, by using the corrected fluorescence and the aging level estimation model in operation 740.

Then, the apparatus for estimating an aging level may predict the risk of cardiovascular disease/aging-related disease by using an aging level estimation result in operation 750. For example, if the aging level of blood vessels exceeds a threshold value, the apparatus for estimating an aging level may predict an increase in the risk of cardiovascular disease. Alternatively, upon further considering a user's biological age, if a change in the estimated aging level of blood vessels is greater than or equal to a predetermined threshold value compared to a reference aging level of blood vessels for a biological age group, the apparatus for estimating an aging level may predict a high risk of cardiovascular disease.

Subsequently, the apparatus for estimating an aging level may output and provide an estimation result of the aging level which is estimated in operation 740, the risk of cardiovascular disease/aging-related disease which is predicted in operation 750, predetermined actions in response to the estimation result or prediction result, and the like, by various visual/non-visual methods for a user in operation 760.

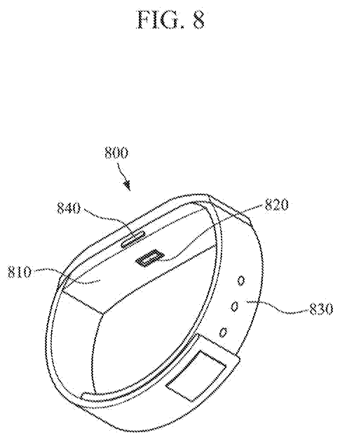

FIG. 8 is a diagram illustrating an example of a wearable device. The apparatus for estimating an aging level according to an example embodiment, described above with reference to FIG. 4, may be mounted in a smart watch worn on a wrist or a smart band-type wearable device.

Referring to FIG. 8, the wearable device 800 includes a main body 810 and a strap 830.

The main body 810 may be formed to have various shapes, and may include various modules which are mounted inside or outside of the main body 810 to perform the aforementioned function of estimating an aging level and various other functions. A battery may be embedded in the main body 810 or the strap 830 to supply power to the various modules of the wearable device 800.

The strap 830 may be connected to the main body 810. The strap 830 may be flexible so as to be wrapped around a user's wrist. The strap 830 may be bent in a manner that allows the strap 830 to be detached from the user's wrist or may be formed as a band that is not detachable. Air may be injected into the strap 830 or an airbag may be included in the strap 830, so that the strap 830 may have elasticity according to a change in pressure applied to the wrist, and the change in pressure of the wrist may be transmitted to the main body 810.

The main body 810 may include a fluorescence measuring sensor 820 for measuring fluorescence of an object. The fluorescence measuring sensor 820 may be mounted on a rear surface of the main body 810, and may include a light source for emitting light to skin of an object such as a wrist, a finger, and the like, and a small spectrometer chip for detecting fluorescence scattered or reflected from the object.

A processor may be mounted in the main body 810, and may be electrically connected to various modules of the wearable device 800 to control operations thereof.

Further, the processor may estimate an aging level by using the fluorescence measured by the fluorescence measuring sensor 820. The processor may correct the measured fluorescence based on an intensity of light incident on the object and an intensity of light reflected from the object, and may estimate an aging level by using the corrected fluorescence. The processor may estimate an aging level from the fluorescence by using an aging level estimation model stored in a storage. Moreover, the processor may predict the risk of cardiovascular disease or aging-related disease of a user by using an aging level estimation result.

In addition, a storage, which stores processing results of the processor and a variety of information, may be mounted in the main body 810. In this case, the variety of information may include reference information for estimating an aging level, as well as information related to the functions of the wearable device 800.

Furthermore, the main body 810 may include a manipulator 840 which receives a user's control command and transmits the received command to the processor. The manipulator 840 may include a power button to input a command to turn on/off the wearable device 800.

The display may be mounted on a front surface of the main body 810, and may be a touch panel for sensing a touch input. The display may receive a touch input from a user, may transmit the received touch input to the processor, and may display a processing result of the processor.

For example, the display may display an aging level estimation result, a prediction result of the risk of cardiovascular disease/aging-related disease, predetermined actions, and the like.

In addition, a communication interface, which communicates with an external device such as a user's mobile terminal, may be mounted in the main body 810. The communication interface may transmit an aging level estimation result and the like to an external device such as a user's smartphone, such that the estimation result may be displayed to the user.

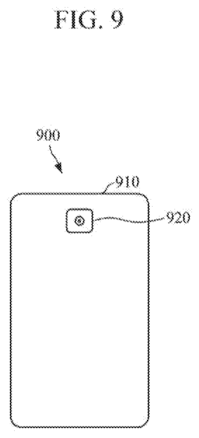

FIG. 9 is a diagram illustrating an example of a smart device. In this case, the smart device may be a smartphone, a tablet PC, and the like.

Referring to FIG. 9, the smart device 900 includes a main body 910 and a sensor 920 mounted on a rear surface of the main body 910. However, the sensor 920 is not limited thereto, and may be mounted on a front surface, a side surface, and the like, without specific limitation on the position. A fluorescence measuring sensor 920 may include one or more light sources and a small spectrometer chip. The light sources may emit blue light to an object, and the small spectrometer chip may detect fluorescence reflected or scattered from the object.

In addition, a display may be mounted on a front surface of the main body 910. The display may visually display an aging level estimation result, a prediction result of the risk of cardiovascular disease/aging-related disease, and the like. The display may include a touch panel, and may receive a variety of information input through the touch panel and transmit the received information to the processor.

The processor may estimate an aging level by using the fluorescence, measured by the fluorescence measuring sensor 920, and an aging level estimation model. Furthermore, based on the aging level estimation result, the processor may further predict the risk of cardiovascular disease or the risk of aging-related disease, and may visually output the prediction result through the display.

The main body 910 of the smart device 900 may include a storage which stores reference information for operation of the smart device 900, including information input by a user, information obtained by various sensors, information processed by the processor, reference information required for estimating an aging level, and the like.

Further, the main body 910 of the smart device 900 may include a communication interface for communication with various external devices, e.g., a wearable device, a desktop computer, a laptop computer, a tablet PC, an external apparatus for measuring fluorescence, a smart device of other users, and the like.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating an aging level, the apparatus comprising:
a light emitter configured to emit blue light to an object;
a light receiver configured to measure fluorescence emanating from the object; and
a processor configured to estimate an aging level of the object based on the measured fluorescence and an aging level estimation model.

2. The apparatus of claim 1, wherein the processor is further configured to correct the measured fluorescence based on either one or both of a first intensity of the blue light emitted from the light emitter and a second intensity of the blue light reflected from the object.

3. The apparatus of claim 1, wherein the light receiver comprises either one or both of a spectrometer and an image sensor.

4. The apparatus of claim 3, wherein the light receiver further comprises a high-pass filter configured to pass a light beam having a wavelength of 500 nm or greater, among a plurality of light beams emanating from the object, and
wherein the at least one of the spectrometer and the image sensor is configured to measure the fluorescence based on the light beam having the wavelength of 500 nm or greater.

5. The apparatus of claim 1, wherein the light emitter is further configured to emit the blue light having a single wavelength or a plurality of wavelengths.

6. The apparatus of claim 1, wherein the light emitter comprises:
a light source configured to emit a light, and
a low-pass filter or a band-pass filter, the low-pass filter being configured to pass a wavelength of 500 nm or less of the light emitted from the light source, the band-pass filter being configured to pass a wavelength range of 400 nm to 500 nm of the light emitted from the light source, and
wherein the light emitter is further configured to emit the light having the wavelength of 500 nm or less, or the light in the wavelength range of 400 nm to 500 nm, as the blue light.

7. The apparatus of claim 1, wherein the aging level estimation model comprises a regression model which defines at least one of a first correlation between the fluorescence and a biological age and a second correlation between the fluorescence and the aging level of blood vessels.

8. The apparatus of claim 7, wherein the processor is further configured to estimate either one or both of the biological age and the aging level of blood vessels by using the regression model.

9. The apparatus of claim 1, wherein the processor is further configured to predict either one or both of a risk of cardiovascular disease and a risk of aging-related disease of a user based on the estimated aging level.

10. The apparatus of claim 9, further comprising an output interface configured to output at least one of the estimated aging level, the risk of cardiovascular disease, and the risk of aging-related disease.

11. A method of estimating an aging level, the method comprising:
emitting blue light to an object;
measuring fluorescence emanating from the object;
estimating an aging level of the object based on the measured fluorescence and an aging level estimation model.

12. The method of claim 11, wherein the estimating the aging level comprises correcting the measured fluorescence based on either one or both of a first intensity of the blue light emitted to the object and a second intensity of the blue light reflected from the object.

13. The method of claim 11, further comprising passing a light beam having a wavelength of 500 nm or greater, among a plurality of light beams emanating from the object, and
wherein the measuring the fluorescence comprises measuring the fluorescence based on the light beam having the wavelength of 500 nm or greater.

14. The method of claim 11, wherein the emitting the blue light comprises, by using a light source, emitting blue light having a single wavelength or a plurality of wavelengths.

15. The method of claim 11, wherein the emitting the blue light comprises:
emitting a light by a light source;
passing a wavelength of 500 nm or less of the light emitted from the light source, or passing a wavelength range of 400 nm to 500 nm of the light emitted from the light source; and
emitting, as the blue light, the light having the wavelength of 500 nm or less, or the light in the wavelength range of 400 nm to 500 nm.

16. The method of claim 11, further comprising predicting either one or both of a risk of cardiovascular disease and a risk of aging-related disease of a user based on the estimated aging level.

17. The method of claim 16, further comprising outputting at least one of the estimated aging level, the risk of cardiovascular disease, and the risk of aging-related disease.

18. An apparatus for estimating an aging level, the apparatus comprising:
a fluorescence measuring sensor comprising a plurality of light sources configured to emit blue light to an object, and a spectrometer configured to measure fluorescence emanating from the object; and
a processor configured to estimate an aging level of the object based on the measured fluorescence.

19. The apparatus of claim 18, wherein the processor is further configured to correct the measured fluorescence based on either one or both of a first intensity of the blue light emitted from the fluorescence measuring sensor and a second intensity of the blue light reflected from the object, and estimates the aging level based on the corrected fluorescence and an aging level estimation model.

20. The apparatus of claim 18, further comprising an output interface configured to output the estimated aging level.
